(19) United States
(12) Patent Application Publication
(10) Pub. No.: US 2022/0313704 A1
(43) Pub. Date: Oct. 6, 2022

(54) COMPOSITIONS CONTAINING ANDROGEN RECEPTOR ANTAGONISTS

(71) Applicant: Chemistry RX, Folcroft, PA (US)

(72) Inventor: Lars Brichta, Brooklyn, NY (US)

(21) Appl. No.: 17/711,105

(22) Filed: Apr. 1, 2022

Related U.S. Application Data
(60) Provisional application No. 63/200,906, filed on Apr. 2, 2021.

(51) Int. Cl. A61K 31/573; A61K 31/167; A61K 31/277; A61K 31/4166; A61K 31/585; A61K 31/496; A61K 31/4164; A61K 31/137; A61K 31/506; A61K 31/517; A61K 31/522; A61K 31/4422; A61K 31/58; A61K 31/519; A61K 31/436; A61K 31/5575; A61K 31/655; A61K 31/4045; A61P 17/14

(52) U.S. Cl. CPC A61K 31/573; A61K 31/167; A61K 31/277; A61K 31/4166; A61K 31/585; A61K 31/496; A61K 31/4164; A61K 31/137; A61K 31/506; A61K 31/517; A61K 31/522; A61K 31/4422; A61K 31/58; A61K 31/519; A61K 31/436; A61K 31/5575; A61K 31/655; A61K 31/4045; A61P 17/14

(57) ABSTRACT

Compositions and methods for treating hair loss, for example hair loss associated with acne related to hormonal conditions, using topically administered androgen receptor antagonists are described herein. The androgen receptor antagonist may be one selected from cyproterone acetate, flutamide, RU-58841, bicalutamide, nilutamide, canrenone, spironolactone, progesterone, 4MA, ketoconazole, cimetidine, and derivatives and combinations thereof.

### CROSS-REFERENCE TO RELATED APPLICATIONS

This application claims priority to and benefit of U.S. Pat. App. No. 63/200,906, entitled “Compositions Containing Androgen Receptor Antagonists,” filed Apr. 2, 2021, the disclosure of which is incorporated by reference herein, in its entirety and for all purposes.

### BACKGROUND

Alopecia, or baldness, is a common diagnosis in clinical practice. Male and female pattern hair loss, sometimes described as “Androgenetic Alopecia” is the most common cause of hair loss. Improved compositions and methods of treating hair loss are needed.

### SUMMARY OF THE INVENTION

Disclosed are compositions and methods for treating female pattern baldness. In one aspect, the method may comprise topically administering a composition comprising, for example, up to about 10% (w/w) androgen receptor antagonist to an individual in need thereof.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g. “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g., animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, aryl aliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylate, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethane sulfonic, sulfanilic, cyclohexyl aminosulfonic, alginic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to topical compositions containing one or more androgen receptor antagonists for treating hair loss and various skin conditions. Other embodiments are directed to methods for treating hair loss and various skin conditions that include administering a topical composition containing one or more androgen receptor antagonists to a subject in need of treatment. In some embodiments, hair loss may be associated with female pattern baldness or androgenic alopecia. The skin conditions encompassed by various embodiments may or may not be associated with endocrine conditions that contribute to or are the underlying cause of female pattern baldness and include, for example, acne.

The androgen receptor antagonists of various embodiments may be any compound known in the art. The androgen receptor is a type of nuclear receptor that is activated by binding any of the androgenic hormones or steroids such as testosterone and dihydrotestosterone and in the cytoplasm and then translocating into the nucleus. The androgen receptor is a DNA-binding transcription factor that regulates gene expression many of which are critical for the development and maintenance of the male sexual phenotype. Despite being linked to androgenic alopecia, many female hair loss sufferers do not exhibit elevated male hormones, and treatment with androgen receptor antagonists or steroid enzyme inhibitors does not typically restore hair growth.

In some embodiments, the androgen receptor antagonist may be, for example, cyproterone acetate, flutamide, RU-58841, bicalutamide, nilutamide, canrenone, spironolactone, progesterone, 4MA, ketoconazole, cimetidine, and the like, derivatives thereof, and combinations thereof. The concentration of androgen receptor antagonists in such embodiments can be up to about 10% (w/w) of one or more androgen receptor antagonist. For example, in some embodiments, the composition may include from about 0.1% (w/w) to about 15% (w/w), from about 0.5% (w/w) to about 10% (w/w), from about 0.75% (w/w) to about 7.5% (w/w), from about 1% (w/w) to about 5% (w/w), from about 1% (w/w) to about 3% (w/w), or any range or individual concentration of androgen receptor antagonist encompassed by these example ranges. In particular embodiments, the composition may include from about 0.25% (w/w) to about 5% (w/w) canrenone, spironolactone, progesterone, or combinations thereof, and in some embodiments, the compositions may include from about 0.25% (w/w) to about 5% (w/w) canrenone or derivatives thereof.

In some embodiments, the compositions may include additional active agents such as, for example, vasodilators, minoxidil, diazoxide, prazosin, nicotinic acid and its esters, caffeine VIP (vasoactive intestinal peptide), maxadilan, nitroglycerin, isosorbide dinitrate, calcium channel blockers, such as nifedipine, finasteride, dutasteride, tofacitinib, tacrolimus, bimatoprost, latanoprost, al dactone, kenalog-10, kenalog-40, tri am ci nol one, azul fi di ne, sulfasalazine, sulfazine, and the like and combinations thereof. Such active agents can be provided in any amount capable of providing treatment. For example, the compositions of embodiments may include up to about 15% (w/w), about 0.25% (w/w) to about 15% (w/w), from about 0.5% (w/w) to about 10% (w/w), from about 0.75% (w/w) to about 7.5% (w/w), from about 1% (w/w) to about 5% (w/w), from about 1% (w/w) to about 3% (w/w), or any range or individual concentration of active agent encompassed by these example ranges, and the like and combinations thereof.

The compositions of various embodiments can be in any form, and embodiments include androgen receptor antagonist containing creams, lotions, foams, liniments, balms, ointments, soaps, shampoos, and the like.

Creams refer to semi-solid emulsions of oil and water in approximately equal proportions. They are divided into two types: oil-in-water (O/W) creams, composed of small droplets of oil dispersed in a continuous phase; and water-in-oil (W/O) creams, composed of small droplets of water dispersed in a continuous oily phase. Creams can provide a barrier to protect the skin. This may be a physical barrier or a chemical barrier as with UV-absorbing compounds. To aid in the retention of moisture (especially water-in-oil creams), creams are usually used for a variety of purposes including cleansing, emollient effects, and as a vehicle for drug substances such as local anesthetics, anti-inflammatories (NSAIDs or corticosteroids), hormones, antibiotics, antifungals or counter-irritants.

Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

Ointments are compositions in which oil and water are provided in a ratio of from 7:1 to 2:1, from 5:1 to 3:1, or 4:1. Ointments are generally formulated using oils, waxes, water, alcohols, petroleum products, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

In some embodiments, the formulations can be in the form of a soap, which are formulations that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerine) is usually liberated and is either left in or washed out and recovered as a useful byproduct, depending on the process employed.

In certain embodiments, the compositions may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, and the like and combinations thereof. In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™ PCCA. Such liposomal base formulations can include, for example, about 60-80% wt/wt water combined with glycerin, C₁₂₋₁₅ alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), prunus amygdalus amara (bitter almond) kernel oil, Vitis vinifera (Grape) seed extract, Triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof. In particular embodiments, the base may be hydrophilic petrolatum, hydroxystearic sulfate, or anhydrous lanolin.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 65% (w/w) to about 90°/% (w/w) of the total composition, or any range or individual concentration known in the art.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include an anti-acne compound such as, for example, salicylic acid and benzoyl peroxide. The amount of the anti-acne compound in the topical formulation is not particularly limited, so long as it is a therapeutically effective amount, typically, about 0.01% (w/w) to 5% (w/w) of the total composition.

In some embodiments, the compositions may further include a humectant that provides soothing, smoothing, moisturizing, or protects the skin. The humectant is not limited and can be, for example, calamine, dodecyl sulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctyl sulfosuccinate (DOSS), lecithin, and sodium docusate. The amount of humectant in such compositions may be about 0.01% (w/w) to 5% (w/w) of the total composition.

In some embodiments, the composition may further include a UV-absorbing compound such as, for example, glyceIyl PABA, padimate 0, roxadimate, dioxybenzone, oxybenzone, sulisobenzone, octocrylene, octyl methoxycinnamate, ethoxyethyl p-methoxycinnamate, homomenthyl salicylate, ethylhexyl salicylate, trolamine salicylate, avobenzone, ecamsule, ensulizole, bemotrizinol, bisoctrizole, and the like and combinations thereof. The amount of UV-absorbing compound may be about 0.01% (w/v) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

Particular compositions encompassed by the various embodiments described above include 0.6% to 2% tofacitinib, DMSO, hydrophilic petrolatum, sorbitan monooleate, and an emulsion stabilizer such as Emulsifix-205; 06% to 2% tofacitinib citrate, DMSO, hydrophilic petrolatum, sorbitan monooleate; and an emulsion stabilizer; 0.6% to 2% ruxolitinib, DMSO, hydrophilic petrolatum, sorbitan monooleate, and an emulsion stabilizer, and 0.6% to 2% ruxolitinib citrate, DMSO, hydrophilic petrolatum, sorbitan monooleate; and an emulsion stabilizer.

Penetration enhancers can have various forms. For example, in some embodiments, the penetration may be a carrier and vehicle such as microcapsules or nanocapsules, nanoemulsions, submicron emulsions, miniemulsions, solid lipid nanoparticles, multiple emulsions, microemulsions, liposomes, niosomes, transfersomes (i.e. vesicles composed of phospholipids and 10% to 25% surfactant such as sodium cholate and 3% to 10% ethanol), ethosomes, aquasomes, and the like and combinations thereof. In other embodiments, the penetration enhancer may be a chemical penetration enhancer such as a sulphoxide, for example, dimethyl sulphoxide (DMSO), decyl methyl sulfoxide, azone (1-dodecyl acyclic heptan-2-one or laurocapram), pyrrolidones such as N-methyl pyrrolidone, 2-pyrrolidone and N-cyclohexyl-2-pyrrolidone, and mixtures thereof, oxazolidinones, and urea. In further embodiments, the penetration enhancers may be diols such as 1,2-hexanediol, butylene glycol, diethylene glycol, dipropylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol and polypropylene glycol, and mixtures thereof or polyols such as butanetriol, glycerol and 1,2,6-hexanetriol, and mixtures thereof. In still other embodiments, the penetration enhancers may be fatty acids such as essential oil, terpenes, terpenoids, oleic acid, capric acid, hexanoic acid, lauric acid, linoleic acid, linolenic acid, propionic acid and vaccenic acid, and mixtures thereof. In some embodiments, the penetration enhancers may be a fatty alcohol such as cetyl alcohol, stearyl alcohol, decanol, tridecanol, lauryl alcohol, linolenyl alcohol and oleyl alcohol, and mixtures thereof or a fatty acid ester such as glycerol monolaurate, glycerol monooleate, glycerol monolinoleate, isopropyl isostearate, isopropyl palmitate, isopropyl myristate, diethyl sebacate, sorbitan monopalmitate, sorbitan oleate, sorbitan dilaurate, sorbitan trioleate, propylene glycol monolaurate and sucrose monolaurate, and mixtures thereof. In further embodiments, the penetration enhancer may be a surfactant. In still further embodiments the penetration enhancer may be a combination of one or more classes of penetration enhancer.

The compositions of the various embodiments described herein may include one or more penetration enhancers. The penetration enhancer in the compositions of various embodiments described above may be present in an amount about 0.5% (w/w) to about 40% (w/w), about 1% (w/w) to about 20% (w/w), about 5% (w/w) to about 15% (w/w) based on the total composition or any range or individual concentration encompassed by these example ranges.

In certain embodiments, the compositions may be a topical cream or solution including an androgen receptor antagonist, a vasodilator, a stimulant, and a prostaglandin. Examples of such compositions may include about 0.5% (w/w) to about 3% (w/w) androgen receptor antagonist, about 3% (w/w) to about 8% (w/w) vasodilator, about 0.05% (w/w) to about 1% (w/w) stimulant, and about 0.005% (w/w) to about 0.5% (w/w) prostaglandin, and particular examples include about 0.5% (w/w) to about 3% (w/w) canrenone, about 3% (w/w) to about 8% (w/w) minoxidil, about 0.05% (w/w) to about 1% (w/w) caffeine, and about 0.005% (w/w) to about 0.5% (w/w) latanoprost. In some embodiments, the composition may be a topical cream or solution including about 5% (w/w) minoxidil, about 1.5% (w/w) canrenone, about 0.2% (w/w) caffeine, about 0.1% (w/w) melatonin, and about 0.02% (w/w) latanoprost solution.

Other embodiments of the invention include methods for treating hair loss or skin conditions by administering any of the compositions described above. Such methods are not limited to particular indications; however, the compositions described herein can be particularly useful for treating hair loss may be associated with female pattern baldness or androgenic alopecia, and/or skin conditions associated with endocrine conditions that contribute to or are the underlying cause of female pattern baldness and including, for example, acne.

The methods of such embodiments may include the steps of administering a composition of the various embodiments described above to the skin of subject in need of treatment topically by, for example, applying the composition to affected areas such as the scalp or skin having lesions or sores associated with acne. The step of administering can be carried out one, two, three, four, or more times per day, and administering can be carried out the prescribed number of times per day for one or more days, one or more weeks, one or more months, one or more years or indefinitely. In some embodiments, administering can be carried out until the symptoms associated with the underlying conditions have been reduced or eliminated.

The step of administering can be carried out by various means. For example, administering can be accomplished by applying the composition to the skin of a subject, and in some embodiments, the skin may be massaged or rubbed to facilitate uptake of the androgen receptor antagonist and other active ingredients. In some embodiments, administering may include applying mechanical force or energy to the skin of the subject to facilitate uptake of the androgen receptor antagonist and other active ingredients. For example, administering includes injecting the composition into the skin of the subject using microneedles. In other embodiments, administering may accomplished using electroporation, iontophoresis, ultrasound, laser radiation and photomechanical waves, magnetophoresis, thermophoresis, radio frequency, suction ablation, skin abrasion and the like and combinations thereof. As is known in the art, certain means for administering may require the use of particular components of the formulation. Such components are described above and can be appropriately incorporated into the compositions.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

A composition for treating female pattern baldness is provided in Table 1

TABLE 1

| Ingredient | Amount |
|---|---|
| minoxidil | 5% |
| canrenone | 1.5% |
| caffeine | 0.2% |
| melatonin | 0.1% |
| latanoprost | 0.02% |

The composition of Table 1 was formulated as a solution and was administered directly to the scalp of a post-menopausal female patient diagnosed exhibiting female pattern baldness twice daily. The patient reported thickening of hair and increased number of active hair follicles after three months of treatment.

What is claimed is:

1. A method for treating female pattern baldness comprising topically administering a composition comprising up to about 10% (w/w) androgen receptor antagonist to an individual in need thereof.

2. The method of claim 1, wherein the androgen receptor antagonist is selected from cyproterone acetate, flutamide, RU-58841, bicalutamide, nilutamide, canrenone, spironolactone, progesterone, 4MA, ketoconazole, cimetidine, and derivatives and combinations thereof.

3. The method of claim 1, said composition comprising from about 0.25% (w/w) to about 5% (w/w) androgen receptor antagonist.

4. The method of claim 1, said composition comprising one or more additional agent selected from a vasodilator, minoxidil, diazoxide, prazosin, nicotinic acid and its esters, caffeine, VIP (vasoactive intestinal peptide), maxadilan, nitroglycerin, isosorbide dinitrate, a calcium channel blocker, such as nifedipine, finasteride, dutasteride, tofacitinib, tacrolimus, bimatoprost, latanoprost, aldactone, kenalog-10, kenalog-40, triamcinolone, azulfidine, sulfasalazine, sulfazine, and combinations thereof

5. The method of claim 1, said composition further comprising a base.

6. The method of claim 1, said composition being formulated as a solution.

7. A composition comprising up to about 10% (w/w) of an androgen receptor antagonist.

8. The composition of claim 7, said androgen receptor antagonist being selected cyproterone acetate, flutamide, RU-58841, bicalutamide, nilutamide, canrenone, spironolactone, progesterone, 4MA, ketoconazole, cimetidine, and derivatives and combinations thereof.

9. The composition of claim 7, said composition comprising from about 0.25% (w/w) to about 5% (w/w) androgen receptor antagonist.

10. The composition of claim 7, further comprising an additional agent selected from a vasodilator, minoxidil, diazoxide, prazosin, nicotinic acid and its esters, caffeine, VIP (vasoactive intestinal peptide), maxadilan, nitroglycerin, isosorbide dinitrate, calcium channel blockers, such asnifedipine, finasteride, dutasteride, tofacitinib, tacrolimus, bimatoprost, latanoprost, aldactone, kenalog-10, kenalog-40, triamcinolone, azulfidine, sulfasalazine, sulfazine, and combinations thereof.

11. The composition of claim 7, further comprising a base.

12. The composition of claim 7, said composition being formulated as a solution.
